(19) United States
(12) Patent Application Publication
(10) Pub. No.: US 2003/0208790 A1
(43) Pub. Date: Nov. 6, 2003

(54) Insect resistant plants and methods for making same

(76) Inventors: Stefan Jansens, Gent (BE); Sara Van Houdt, Zottegem (BE); Arlette Reynaerts, Drongen (BE)

Correspondence Address: BURNS DOANE SWECKER & MATHIS L L P, POST OFFICE BOX 1404, ALEXANDRIA, VA, 22313-1404, US

(21) Appl. No.: 10/137,682

(22) Filed: May 3, 2002

(51) Int. Cl. A01H 1/00; C12N 15/82; C07H 21/04; C12N 5/04

(52) U.S. Cl. 800/279; 435/468; 435/419; 435/320.1; 536/23.6

(57) ABSTRACT

The present invention relates to a DNA sequence encoding a modified Cry1Ab protein that has insecticidal activity. The invention further relates to a method for producing insect resistant plants by introducing into the genome of the plants a foreign DNA comprising such a modified cry1Ab coding sequence. The invention further relates to plants or parts thereof comprising in their genome the modified cry1Ab coding sequence of the present invention.

### FIELD OF THE INVENTION

The present invention relates to a DNA sequence encoding a modified Cry1Ab protein that has insecticidal activity. The invention further relates to a method for producing insect resistant plants by introducing into the genome of the plants a foreign DNA comprising such a modified cry1Ab coding sequence. The invention further relates to plants or parts thereof comprising in their genome the modified cry1Ab coding sequence of the present invention.

### BACKGROUND ART

The Gram-positive soil bacterium Bacillus thuringiensis is well known for its production of proteins or delta-endotoxins, that are toxic to a variety of lepidopteran, coleopteran, and dipteran larvae. Different strains of B. thuringiensis have been shown to produce different insecticidal crystal proteins, which are specifically toxic to certain species of insects (reviewed by Höfte et al. 1989; Schnepfet al., 1998).

The specific toxicity of insecticidal toxins produced by B. thuringiensis for target insects and their non-toxicity to plants and other organisms has made compositions comprising different Bt strains the product of choice for the biological control of agricultural insect pests. Various of the genes encoding the crystal proteins have been cloned and their DNA sequences determined (Höfte and Whiteley, 1989; Crickmore et al., 1995). This has led to the engineering of modified delta-endotoxin encoding genes and the development of plants expressing these delta-endotoxin genes to make them insect resistant.

The family of cry1genes encodes the Cry1crystal proteins, which are primarily active against lepidopteran pests. The pro-toxin form of Cry1delta-endotoxins consists of a C-terminal protoxin part, which is not toxic and is thought to be important for crystal formation (Arvidson et al., 1989). The amino-half of the protoxin comprises the active toxin segment of the Cry1protein. Different domains have further been identified in the active toxin that are implcatied in different aspects of the toxicity effect (Grochulski et al., 1995). However, these functions seem to be dependent on the delta endotoxin examined.

Significant effort has gone into modifying the cry1genes to improve expression levels in plants while at least retaining their toxicity to the target insects. Modification of the cry1Ab and cry1Ac genes to remove putative plant polyadenylation signals and instability motifs (without altering the encoded amino acid sequences) resulted in increased resistance of the plants transformed with these sequences (van der Salm et al., 1994). Modifications of the cry1Ab gene have been described in U.S. Pat. No. 6,320,100, U.S. Pat. No. 6,180,774 and U.S. Pat. No. 6,114,608. U.S. Pat. No. 5,500,365 describes how modification in the 240 region of the coding region of a cry1Ab gene so as to remove putative plant signals is of significant importance to increase expression levels and thereby toxicity of the Cry toxin in plants.

The present invention relates to a novel modified Cry1Ab protein and DNA sequences encoding this protein, which can be used to engineer insect resistance in plants. More particularly, it was found that this modified sequence, despite having a native 240 region, ensures sufficiently high expression in plant cells to confer insect resistance to the plant or plant tissue in which it is expressed.

### SUMMARY OF THE INVENTION

The present invention relates to a modified cry1Ab coding sequence, which encodes the modified Cry1Ab protein of SEQ ID NO: 1, which is an insecticidal protein. According to a particular embodiment of the invention, the DNA sequence encoding the modified Cry1Ab sequence corresponds to the sequence of SEQ ID NO: 2.

The invention further relates to chimeric genes comprising the modified cry1Ab DNA sequence of the present invention under the control of a plant-expressible promoter. According to a particular embodiment of the present invention the plant-expressible promoter is either a constitutive promoter, a tissue-specific promoter or a wound-inducible promoter or a promoter that ensures expression of the modified Cry1Ab protein at least in the cells or tissues of a plant which are susceptible to insect attack.

The invention further relates to recombinant vectors comprising the chimeric genes of the invention and to the production of transgenic plants using these recombinant vectors.

The invention further relates to plants and cells, seeds or tissues thereof, comprising in their genome a foreign DNA comprising the modified cry1Ab DNA sequence of the present invention under the control of a plant-expressible promoter.

The invention also relates to a method for engineering insect resistance in plants, by introducing, into the genome of the plant, a foreign DNA comprising the modified cry1Ab coding sequence of the present invention under the control of a plant-expressible promoter.

According to a particular embodiment of the present invention the modified cry1Ab coding sequence is particularly suited for engineering insect resistance in agricultural crops such as corn and cotton. Most particularly, expression of the modified Cry1Ab protein confers resistance to lepidopteran pests of these plants. More particularly, these pests include, but are not limited to, major lepidopteran pests of corn, cotton and rice, such as Ostrinia nubilalis (European corn borer or ECB), Sesamia nonagroides (Mediterranean Stalk borer), Sesamia inferens (Pink stemborer), Helicoverpa zea (corn earworm, cotton bollworm), Helicoverpa armigera (American bollworm), Heliothis viriscens (Tobacco budworm), Scirpophaga incertulas (Yellow stemborer), Sesamia inferens (pink stem borer) and Cnaphalocrosis medinalis (Rice leaf folder).

### DETAILED DESCRIPTION

The term “gene” as used herein refers to any DNA sequence comprising several operably linked DNA fragments such as a promoter region, a 5′ untranslated region (the 5′UTR), a coding region, and an untranslated 3′ region (3′UTR) comprising a polyadenylation site. A gene may include additional DNA fragments such as, for example, introns.

The term “chimeric” when referring to a gene or DNA sequence is used to refer to the fact that the gene or DNA sequence comprises at least two functionally relevant DNA fragments (such as promoter, 5′UTR, coding region, 3′UTR, intron) that are not naturally associated with each other and/or originate, for example, from different sources. “Foreign” referring to a gene or DNA sequence with respect to a plant species is used to indicate that the gene or DNA sequence is not naturally found in that plant, or is not naturally found in that genetic locus in that plant. The term “foreign DNA” will be used herein to refer to a DNA sequence as it has incorporated into the genome of a plant as a result of transformation.

A genome of a plant, plant tissue or plant cell, as used herein, refers to any genetic material in the plant, plant tissue or plant cell, and includes both the nuclear and the plastid and mitochondrial genome.

A “fragment” or “truncation” of a DNA molecule or protein sequence as used herein refers to a portion of the original DNA or protein sequence (nucleic acid or amino acid) referred to or a synthetic version thereof (such as a sequence which is adapted for optimal expression in plants), which can vary in length but which is sufficient to ensure the (encoded) protein to be an insect toxin. A “variant” of a sequence is used herein to indicate a DNA molecule or protein of which the sequence (nucleic or amino acid) is essentially identical to the sequence to which the term refers.

Sequences which are “essentially identical” means that when two sequences are aligned, the percent sequence identity, i.e. the number of positions with identical nucleotides or amino acids divided by the number of nucleotides or amino acids in the shorter of the sequences, is higher than 70%-80%, preferably 81-85%, more preferably 86-90%, especially preferably 91-95%, 10 most preferably 96-100%. The alignment of two nucleotide sequences is performed by the algorithm as described by Wilbur and Lipmann (1983, Proc. Natl. Acad. Sci. U.S.A. 80:726) using a window size of 20 nucleotides, a word length of 4 nucleotides, and a gap penalty of 4.

A ‘plant-expressible promoter’ as used herein refers to a promoter which ensures expression of a coding sequence to which it is linked in a plant cell. Examples of such promoters are well known in the art. A plant-expressible promoter can be a constitutive promoter. Examples of promoters directing constitutive expression in plants are known in the art and include the 35S promoter from Cauliflower Mosaic virus, the nopaline synthase (NOS) promoter, the ubi promoter (Christensen et al. 1992), the promoter of the GOS2 gene from rice (de Pater et al., 1992). Alternatively, a plant-expressible promoter can be a tissue-specific promoter, i.e., a promoter directing a higher level of expression of a coding sequence (as can be measured by conventional RNA assays) in some tissues of the plant, e.g. in green tissues (such as the promoter of the PEP carboxylase) than in other tissues of the plant. Alternatively, a plant-expressible promoter can be a wound-inducible promoter. A ‘wound-inducible’ promoter or a promoter directing an expression pattern that is wound-inducible, as used herein means that upon wounding of the plant, either mechanically or by insect feeding, expression of the coding sequence under control of the promoter is significantly increased. Examples of wound-inducible promoters include the proteinase inhibitor gene of potato and tomato (pin1 and pin2)(Johnson et al., 1989) and the promoter of the maize proteinase inhibitor (MPI) gene (Cordero et al. 1994). The ‘TR2′promoter’ as used herein relates to a promoter region comprising the TR2′ (or mas) functional part of the TR1-TR2 dual promoter element from Agrobacterium (Velten et al. 1984; Langridge et al. 1989). Thus this can comprise the TR2′ element either alone or in combination with the divergent TR1 element (Guevara-Garcia et al., 1998).

‘Insecticidal’ is used herein to mean toxic to insects that are crop pests. More particularly, in the context of the present invention target insects are the pests such as, but not limited to major lepidopteran pests, such as Ostrinia nubilalis (European corn borer or ECB), Sesamia nonagroides (Mediterranean Stalk borer), Helicoverpa zea (corn earworm, cotton bollworm), Helicoverpa armigera (American bollworm) and Heliothis viriscens (Tobacco budworm).

In a preferred embodiment of the present invention, the DNA encoding an insecticidal crystal protein (ICP) is a modified cry1Ab DNA sequence encoding an ICP that is a modified Cry1Ab protein. According to a preferred embodiment, the modified cry1Ab coding sequence encodes the modified Cry1Ab protein corresponding to the sequence of SEQ ID NO: 1. According to a further preferred embodiment of the invention the modified cry1Ab coding sequence corresponds to the sequence of SEQ ID NO: 2.

In a preferred embodiment of the invention, the modified Cry1Ab protein is toxic to major lepidopteran pests of crops such as corn, cotton and rice. The plants of the present invention, comprising a foreign DNA in their genome comprising a DNA encoding a modified Cry1Ab protein are protected against these pests, by expressing a controlling amount of this protein. By controlling is meant a toxic (lethal) or combative (sublethal) amount. At the same time, the plants should be morphologically normal and may be cultivated in a usual manner for consumption and/or production of products. Furthermore, said plants should substantially obviate the need for chemical or biological insecticides (to insects targeted by the modified Cry1Ab protein).

The expression level of an ICP in plant material can be determined in a number of ways described in the art, such as by quantification of the MRNA encoding the insecticidal protein produced in the tissue using specific primers (such as described by Cornelissen & Vandewiele, 1989) or direct specific detection of the amount of insecticidal protein produced, e.g., by immunological detection methods. More particularly, according to the present invention the expression level of a modified Cry1Ab protein is represented as the percentage of soluble insecticidal protein as determined by immunospecific ELISA as described herein related to the total amount of soluble protein (as determined, e.g., by Bradford analysis).

Different assays can be used to measure the insecticidal effect or efficacy of ICP expression in the plant. According to the present invention, the target insects are the major lepidopteran pests of agricultural crops such as corn, cotton and rice, more particularly the European Corn Borer (ECB) and Sesamia nonagroides (SMG) in corn, the cotton bollworm (CBW) and tobacco budworm (TBW) in cotton, and the yellow stem borer, the pink stem borer and the rice leaf folder in rice. The toxicity of an ICP produced in a corn plant on ECB can be assayed in vitro by testing of protein extracted from the plant in feeding bioassays with ECB larvae or by scoring mortality of larvae distributed on leaf material of transformed plants in a petri dish (both assays described by Jansens et al., 1997), or on plants isolated in individual cylinders. In the field first brood European corn borer (ECB1) infestation is evaluated based on leaf damage ratings (Guthrie, 1989) while evaluation of the total number of stalk tunnels per plant and stalk tunnel length are indicative of second brood (ECB2) stalk feeding damage.

Efficacy of the ICP produced in cotton plants transformed with a modified cry1Ab gene can similarly be measured using in vitro and/or in vivo assays. Toxicity of the transformed plant tissue to CBW larvae can be measured by feeding CBW larvae on squares, leaves or terminals and assaying weight of surviving larvae. In the field, plants are artificially infested with neonate CBW larvae and rating damage to leaves, terminals, squares, white bloom and bolls at regular intervals (as described herein). It will be understood that similar assays can be developed for any target or non-target insect in order to determine efficacy of the ICP produced in the plant against such insect.

The plants of the present invention optionally also comprise in their genome a gene encoding herbicide resistance. More particularly, the herbicide resistance gene is the bar or the pat gene, which confers glufosinate tolerance to the plant, i.e. the plants are tolerant to the herbicide Liberty™. Tolerance to Liberty™ can be tested in different ways. For instance, tolerance can be tested by Liberty™ spray application. Spray treatments should be made between the plant stages V2 and V6 for best results. Tolerant plants are characterized by the fact that spraying of the plants with at least 200 grams active ingredient/hectare (g.a.i./ha), preferably 400 g.a.i./ha, and possibly up to 1600 g.a.i./ha (4X the normal field rate), does not kill the plants. A broadcast application should be applied at a rate of 28-34 oz Liberty™ +3# Ammonium Sulfate per acre. It is best to apply at a volume of 20 gallons of water per acre using a flat fan type nozzle while being careful not to direct spray applications directly into the whorl of the plants to avoid surfactant burn on the leaves. The herbicide effect should appear within 48 hours and be clearly visible within 5-7 days.

Examples of other herbicide resistance genes are the genes encoding resistance to phenmedipham (such as the pmph gene, U.S. Pat. No. 5,347,047; U.S. Pat. No. 5,543,306), the genes encoding resistance to glyphosate (such as the EPSPS genes, U.S. Pat. No. 5,510,471), genes encoding bromoxynyl resistance (such as described in U.S. Pat. No. 4,810,648) genes encoding resistance to sulfonylurea (such as described in EPA 0 360 750), genes encoding resistance to the herbicide dalapon (such as described in WO 99/27116), and genes encoding resistance to cyanamide (such as described in WO 98/48023 and WO 98/56238) and genes encoding resistance to glutamine synthetase inhibitors, such as PPT (such as described in EP-A-0 242 236, EP-A-0 242 246, EP-A-0 257 542).

Introduction of a foreign DNA into a plant cell can be obtained by conventional transformation methods described in the art. Such methods include but are not limited to Agrobacterium mediated transformation (U.S. Pat. No. 6,074,877, Hiei et al., 1997), microprojectile bombardment (as described, for example by Chen et al., 1994; Casas et al., 1995; Christou, 1997, Finer et al., 1999, Vasil et al. 1999), direct DNA uptake into protoplasts (as described, for example by De Block et al. 1989; Poulsen, 1996, Datta et al., 1999), electroporation (D' Halluin et al., 1992, U.S. Pat No. 5,641,665, Bates 1999) or silicon whisker mediated DNA introduction (Dunwell, 1999) or other methods as generally reviewed by Potrykus (1990), Sawahel et al. (1995), Komari et al. (1998), Bogorad (2000) and Newell (2000).

The following non-limiting examples describe the development of a DNA sequence encoding a modified Cry1Ab protein, the construction of chimeric genes comprising this sequence for expression in plants, and insect resistant plants obtained therewith. Unless stated otherwise in the Examples, all recombinant DNA techniques are carried out according to standard protocols as described in Sambrook and Russell (2001) Molecular Cloning: A Laboratory Manual, Third Edition, Cold Spring Harbor Laboratory Press, N.Y., in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R.D.D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR - Basics: From Background to Bench, First Edition, Springer Verlag, Germany.

Throughout the description and Examples, reference is made to the following sequences represented in the sequence listing:

SEQ ID NO: 1: Modified Cry1Ab protein

SEQ ID NO: 2: DNA sequence encoding a modified Cry1Ab protein

### EXAMPLES

1. Development of a modified Cry1Ab gene

The modified Cry1Ab DNA sequence used herein encodes part of the Cry1Ab5 protein described by Höfte et al. (1986) corresponding to amino acid 1 to 616, which has an insertion of an alanine codon (GCT) 3′ of the ATG start codon (AlaAsp2. . . Asp616). The protein sequence of the modified Cry1Ab protein is provided in SEQ ID NO: 1. The sequence of the DNA encoding such a modified Cry1Ab protein is provided in SEQ ID NO: 2.

2. Cry1Ab gene in corn

I. Development of Cry1Ab events in corn

For Agrobacterium transformation of corn, constructs were developed wherein the cry1Ab coding sequence was placed under the control of different promoters: 35S promoter (Odell et al. 1985), the ubi promoter (Christensen et al. 1992), the promoter of the GOS2 gene from rice (de Pater et al., 1992) with the cab22 leader from Petunia (Harpster et al. 1988), the 5′ leader sequence of the GOS2 gene from rice, containing the second exon, the first intron and the first exon of the GOS transcript (de Pater et al., 1992) or a TR2′ promoter region (Velten et al. 1984); all constructs included the 35S-bar gene. Agrobacterium-mediated transformation was done by co-cultivation of type I callus derived from immature embryo's with strain C58C1 (pTiEHA101)(pTTS35)(Agrobacterium C58CIRifR strain cured for pTiC58 harboring the non-oncogenic Ti plasmid pTiEHA101 (Hood et al., 1986) and the plasmids in the table below containing the genes of interest placed between the T-DNA borders. Protoplast transformation was done by PEG mediated transfection of protoplasts prepared from suspension cultures derived from Pa91×H99×HE89 Z15 embryos. The DNA used for transfection was a purified fragment of the plasmids in the table below containing the genes of interest between T-DNA borders.

| | |
|---|---|
| Construct description | Abbreviation |

Agrobacterium
transformation

| | | |
|---|---|---|
| PTSVH0203 | p35S2-GE1-modcry1Ab-3′ocs | p35S-cry1Ab |
| | <>p35S3-bar-3′nos | |
| PTSVH0207 | Pubi1-ubi leader with intron- | pUbi-cry1Ab |
| | modcry1Ab-3′ocs<>p35S3-bar-3′nos | |
| PTSVH0208 | Pgos2-cab22 leader-modcry1Ab- | pGos-cab-cry1Ab |
| | 3′ocs<>p35S3-bar-3′nos | |
| PTSVH0209 | Pgos2-gos leader with intron- | pGos-gos-cry1Ab |
| | modcry1Ab-3′ocs<>p35S3-bar-3′nos | |
| PTSVH0212 | 3′nos-bar-p35S3><Tr2-modcry1Ab- | pTR2-cry1Ab |
| | 3′ocs | |
| Protoplast | | |
| transformation | | |
| PSVH0211 | Pubi1-ubi leader with intron- | pUbi-cry1Ab |
| | cry1Ab53-3′ocs<>p35S3-bar-3′nos | |
| PSVH0213 | Pgos2-gos leader with intron- | pGos-gos-cry1Ab |
| | modcry1Ab-3′ocs<>p35S3-bar-3′nos | |

Regenerated plantlets were selected based on Liberty tolerance and/or measurement of PAT protein levels by ELISA.

II. Evaluation of events

a) general characterization

The Agrobacterium transformants were checked for presence of vector sequence at the left border of the T-DNA. Southern blot analyses were performed with leaf material of the primary transformants (TO).

b) Expression of modified Cry1Ab protein

The events were analyzed in the greenhouse for Cry1Ab expression by detecting soluble modified Cry1Ab protein levels in different tissues by a Cry1Ab sandwich ELISA with a polycondensated IgG fraction of a polyclonal rabbit antiserum against Cry1Ab as first antibody and a monoclonal antibody against Cry1Ab as second antibody.

The expression levels of modified Cry1Ab protein in different tissues of early whorl plants transformed with the p35S-cry1Ab construct is provided in Table 1. Expression in the different progeny plants of one event obtained with p35S-cry1Ab event was found to be constitutive and higher than 0.2%.

TABLE 1
Expression of Cry1Ab in different tissues of the early whorl plant

| | Greenhouse | Cry1Ab in % soluble protein/total protein | | | |
|---|---|---|---|---|---|
| Event | plant no | Leaf cylinder | Leaf base | Leaf blade | Root |
| CP058-2602 | 0042 | 0.21 | 0.24 | 0.73 | 1.4 |
| (construct | 0032 | 1.04 | 0.58 | 1.12 | 3.18 |
| ptsvh0203) | 0033 | 0.89 | 0.39 | 1.14 | 2.39 |
| | 0034 | 1.24 | 0.32 | 1.12 | 2.65 |
| | 0035 | 0.7 | 0.44 | 0.94 | 3.51 |
| | 0039 | 0.35 | 0.39 | 0.88 | 2.48 |
| | 0040 | 0.29 | 0.32 | 0.68 | 1.7 |
| | Average (SD) | 0.674(0.40) | 0.38(0.11) | 0.944(0.19) | 2.47(0.75) |
| | 0036 | 0.04 | 0.05 | 0.27 | 0.81 |
| | 0037 | 0.03 | 0.06 | 0.13 | 0.66 |
| | 0038 | 0.02 | 0.04 | 0.15 | 1.15 |
| | 0041 | 0.04 | 0.12 | 0.13 | 0.49 |
| | 0043 | 0.02 | | 0.08 | 0.78 |
| | 0044 | 0.03 | 0.08 | 0.13 | 0.98 |
| | 0045 | 0.01 | 0.05 | 0.08 | 0.55 |
| | 0031 | 0.02 | 0.11 | 0.11 | 0.57 |
| | Average (SD) | 0.026(0.011) | 0.073(0.03) | 0.135(0.06) | 0.75(0.23) |

The expression levels of Cry1Ab protein in different tissues in early V, R1 and R4-5 stage leaves, R4-5stage kernels and in pollen for plants transformed with the p35S-cry1Ab, pGos-cab-cry1Ab and pTR2-cry1Ab constructs are provided in Table 2. Results are presented as averages of leaf and kernel samples taken from 5 plants and as averages of pollen samples taken from 3 plants (standard deviation in brackets). ICP expression for the one p35S-cry1Ab plant is above 0.1% total soluble protein. The pGos-cab-cry1Ab plants showed around 0.01-0.06% Cry1Ab expression in leaves. The events obtained with the pGos-gos-cry1Ab constructs showed high expression (0.2-0.6% protein) in leaf tissue. Basal expression of protein in leaves of plants transformed with the modified cry1Ab DNA sequence under control of the wound-inducible TR2′ promoter (pTR2′-cry1Ab) was low to undetectable.

TABLE 2
Expression of Cry1Ab protein in different tissues of plants transformed with the Cry1Ab gene

| | | Cry1Ab in % soluble protein/total protein | | | | |
|---|---|---|---|---|---|---|
| | | 5 plants | 5 plants | 3 plants | 5 plants | 5 plants |
| Event | Construct | early V/leaf | R1/leaf | pollen | R4-5/leaf | R4-5/kernel |
| CE048-2602 | p35S-cry1Ab | 0.186(0.066) | 0.54 (0.38) | 0 | 0.41(0.17) | 0.048(0.039) |
| CE104-0202 | pGos-cab-cry1Ab | 0.014(0.0055) | | | 0.036(0.015) | |
| CE1014-0402 | pGos-cab-cry1Ab | 0.032(0.016) | | | 0.041(0.011) | |
| CE122-0806 | pGos-cab-cry1Ab | 0.024(0.015) | 0.10 (0.013) | 0 | 0.058(0.036) | 0(0) |
| CE2162-0402 | pGos-gos-cry1Ab | 1.27(0.82) | | | | |
| CE2168-0202 | pGos-gos-cry1Ab | 0.467(0.23) | | | | |
| CE2168-1218 | pGos-gos-cry1Ab | 0.26(0.026) | | | | |
| CE21612-1402 | pGos-gos-cry1Ab | 0.158(0.067) | 0.256 (0.106) | 0 | 0.153(0.023) | 0.017(0.006) |
| CE21614-0604 | pGos-gos-cry1Ab | 0.682(0.50) | | | | |
| CE21614-0816 | pGos-gos-cry1Ab | 0.327(0.27) | 0.925 (0.198), | 0.036 | 0.44(0.078) | 0.0417(0.021) |
| | | | 1 plant 0.06 | (0.0115) | | |
| CP21614-1606 | pGos-gos-cry1Ab | 0.38(0.153) | | | | |
| WI600-0218 | pTR2-cry1Ab | 0 | 0 | 0 | 0 | 0.002 |
| WI600-0802 | pTR2-cry1Ab | 0 | 0 | 0 | 0.0048(0.0016) | 0.01 |

In a greenhouse study, expression of modified Cry1 Ab protein was determined in leaf samples of plants in V3 stage, and, where available, leaf and pollen of plants in R1 stage, and leaf, stalk and pollen of plants at harvest, for events obtained with the different constructs. Again, the plants with the pGos-gos-cry1Ab constructs showed high Cry1Ab expression in leaves (>0.2% total soluble protein) (irrespective of transformation method used). For these plants, high expression levels were also found in the stalk (0.8% total soluble protein or more average). Events obtained from transformation with the pTR2′-cry1Ab constructs showed low (0.02% total soluble protein or less average) or undetectable expression of Cry1Ab protein in all tissues tested.

c) Inducible Cry1Ab expression

For the events obtained with the pTR2′-cry1Ab construct, studies were performed in the greenhouse to determine the expression of the modified Cry1Ab protein upon mechanical damage. Leaf samples were taken before and 18h after mechanical damage and Cry1Ab protein levels were measured by ELISA (Table 3).

TABLE 3
expression of insecticidal protein in different plant parts before and after mechanical wounding

| | | Cry1Ab in % soluble protein/total protein | | | | | | | | | |
|---|---|---|---|---|---|---|---|---|---|---|---|
| | | V4 stage | | Flowering | | | | | | | |
| Event | | | Leaf | | Leaf | | Root | | Harvest | | |
| (construct) | | Leaf | induced | Leaf | induced | Root | induced | Pollen | Leaf | Stalk | Kernel |
| WI600-0218 | Mean | 0.000 | 0.020 | 0.000 | 0.020 | 0.023 | 0.036 | 0.000 | 0.000 | 0.000 | 0.000 |
| (pTR2′-Cry1Ab) | st.dev. | 0.000 | 0.008 | 0.000 | 0.007 | 0.008 | 0.011 | 0.000 | 0.000 | 0.000 | 0.000 |
| WI606-0406 | Mean | 0.005 | 0.046 | 0.000 | 0.007 | 0.012 | 0.008 | 0.000 | \ | 0.002 | 0.002 |
| (pTR2′-Cry1Ab) | st.dev. | 0.007 | 0.005 | 0.000 | 0.006 | 0.009 | 0.009 | 0.000 | | 0.004 | |
| WI604-1602 | Mean | 0.002 | 0.104 | 0.001 | 0.020 | 0.020 | 0.024 | 0.000 | 0.004 | 0.004 | 0.001 |
| (pTR2′-Cry1Ab) | st.dev. | 0.003 | 0.012 | 0.000 | 0.009 | 0.008 | 0.008 | 0.000 | 0.002 | 0.003 | 0.000 |
| WI606-0802 | Mean | 0.003 | 0.064 | 0.001 | 0.010 | 0.027 | 0.037 | 0.000 | 0.002 | 0.004 | 0.003 |
| (pTR2′-Cry1Ab) | st.dev. | | 0.004 | 0.000 | 0.009 | 0.012 | 0.020 | 0.000 | 0.001 | 0.003 | 0.001 |
| WI606-1206 | Mean | 0.001 | 0.081 | 0.000 | 0.022 | 0.032 | 0.024 | 0.000 | 0.004 | 0.002 | 0.001 |
| (pTR2′-Cry1Ab) | st.dev. | | 0.016 | 0.000 | 0.010 | 0.005 | 0.011 | 0.000 | 0.003 | 0.001 | 0.001 |
| CE048-2402 | Mean | 0.83 | 0.614 | 0.280 | 0.397 | 0.196 | 0.253 | 0.000 | 0.376 | 1.120 | 0.047 |
| (p35S-Cry1Ab) | st.dev. | 0.108 | 0.111 | 0.027 | | 0.133 | 0.083 | 0.000 | 0.098 | 0.175 | 0.014 |
| CE048-2602 | Mean | 0.636 | 0.527 | 0.007 | 0.006 | 0.087 | 0.045 | 0.000 | 0.106 | 0.040 | 0.015 |
| (p35S-Cry1Ab) | st.dev. | 0.16 | 0.046 | 0.002 | 0.002 | 0.044 | 0.007 | 0.000 | 0.011 | 0.013 | 0.004 |
| Control 1 | Mean | 0.000 | 0.000 | 0 | 0 | 0 | 0 | 0 | 0 | 0.001 | 0 |
| (untransformed) | st.dev. | 0.000 | 0.000 | 0 | 0 | 0 | 0 | 0 | 0 | 0.002 | 0 |
| Control 2 | Mean | 0.000 | 0.000 | 0 | 0 | 0 | 0 | 0 | 0 | 0 | 0 |
| (untransformed) | st.dev. | 0.000 | 0.000 | 0 | 0 | 0 | 0 | 0 | 0 | 0 | 0 |
| CE0104-0202 | Mean | 0.022 | 0.023 | 0.008 | 0.017 | 0.023 | 0.016 | 0.0001 | \ | \ | \ |
| (pGos/cab-Cry1Ab) | st.dev | 0.009 | 0.005 | 0.005 | 0.004 | 0.012 | 0.005 | 0.000 | | | |
| CE1014-0402 | Mean | 0.025 | 0.017 | 0.007 | 0.010 | 0.028 | 0.034 | 0.000 | 0.010 | 0.040 | 0.002 |
| (pGos/cab-Cry1Ab) | | 0.005 | 0.003 | 0.001 | 0.003 | 0.017 | 0.013 | 0.000 | 0.003 | 0.020 | 0.001 |

Expression of the Cry1Ab protein was either absent or around the detection limit in leaves stalk and kernels of the different events tested. No expression was found in leaves and pollen in whorl and pollen shedding plant stage. Constitutive expression was seen in the roots. When leaves are mechanically damaged, expression of the modified Cry1Ab protein is induced and goes up to 0.02-0.1%.

d) ECB Efficacy

ECB efficacy trials were performed in the greenhouse and at two different locations in the field. At the same time, plants were evaluated for phytotoxicity effects of the constructs introduced. Table 4 shows the results of efficacy tested for ECB. ECB efficacy is determined by measuring the length of tunnels in cm per stalk for 10 plants and is expressed as the average length (sd in brackets) of tunnels per maximum number of tunnels per plant.

TABLE 4
ECB efficacy trials in the greenhouse and in different
locations in the field

| | Greenhouse | Field 1 | Field 2 |
|---|---|---|---|
| | ECB efficacy | ECB efficacy | Efficacy |
| p35S-cry1Ab | | | |
| CE024-1301 | 1.2(1.47)/2 | 0.4(0.53)/4 | 0.05(0.07)/1 |
| PGos-cab-cry1Ab | | | |
| CE050-0802 | 0(0)/0 | 0(0)/0 | 0(0)/14 |
| CE052-0101 | 0.6(0.84)/2 | 0.37(0.43)/3 | 0.17(0.23)/2 |
| CE052-0205 | 1.6(2.4)/6 | 0.08(0.072)/1 | 0.05(0.07)/1 |
| CE053-0605 | 0.4(0.69)/2 | 0.113(0.1)/1 | 0.047(0.08)/1 |
| | | 0.09(0.085)/1 | 0(0)/0 |
| CE053-0702 | 0.6(0.96)/2 | 0.4(0.53)/5 | 0.055(0.097)1 |
| CE057-0201 | | 0.21(0.26)/1 | 0.2(0.35)/6 |
| CE060-0402 | 0.7(1.64)/5 | 0.91(0.44)4 | 0.4(0.692)/3 |
| CE061-0301 | 0.7(1.16)3 | 0.22(0.39)/4 | 0.0625(0.088)/ |
| | | | 1 |
| CE061-1102 | 2.7(1.76)/5 | 1 | |
| CE061-1402 | | 2.8(4.52)/8 | 0.22(0.47)/2 |
| CE061-1502 | 1.7(0.97)/(3) | 0.08(0.14)/1 | 0(0)/0 |
| CE062-0201 | | 0(0)/0 | 0(0)/0 |
| CE067-0401 | | 0(0)/0 | 0.205(0.113)1 |
| CE068-0903 | | 0.07(0.11)/2 | 0.04(0.0072)/1 |
| pGos-gos-Cry1Ab | | | |
| CE180-0303 | 0(0)/0 | 0(0)/0 | 0(0)/0 |
| CE181-0201 | 0.1(0.32)/1 | 0.36(0.34)/2 | 0(0)/0 |
| CE182-0101 | | 0(0)/0 | 0.07(0.1)/0 |
| CE183-0501 | | 0(0)/0 | 00.056(0.08)/1 |
| CE183-0601 | | 1 | 0(0)/0 |
| CE184-0101 | 0.7(1.06)/3 | 0.51(0.35)/2 | 0(0)/0 |
| CE184-0609 | | 0(0)/0 | 0(0)/0 |
| CE184-0801 | 0.11(0.33)/1 | 0(0)/0 | 0(0)/0 |
| CE186-0203 | | 0(0)/0 | 0.037(0.064)/1 |
| CE187-0302 | | 0.08(0.14)/2 | 0(0)/0 |
| CE187-0408 | 0.1(0.32)/1 | 0(0)/0 | 0.11(0.19)1 |
| CE187-0803 | | 0.096(0.17)/1 | 0.097(0.16)1, |
| | | | 10?? |
| CE193-1002 | | 0.15(0.17)/1 | 0.33(0.58)/4 |
| CE196-0201 | 0.4(1.26)/0 | 0(0)/0 | 0.144(0.14)1 |
| CE197-0102 | | 0(0)/0 | |
| CE198-0401 | 0.9(1.28)/3 | 0.09(0.08)/1 | 0(0)/0 |
| CE198-0802 | 0(0)/0 | 0.17(0.21)/3 | 0.03(0.057)/1 |
| CE198-1401 | 0(0)/0 | 0.047(0.0082)/ | 0(0)/0 |
| | | 1 | |
| CE198-1702 | 0.1(0.32)/1 | 0.55(0.74)/4 | 0(0)/0 |
| CE198-2101 | 0.1(0.32)/1 | 0(0)/0 | 0.085(0.12)1 |
| CE198-2501 | 0(0)/0 | 0(0)/0 | 0(0)/0 |
| PUbi-cry1Ab | | | |
| ACE054-00103 | 158.8(32.3)/200 | 19.0(1.4)30 | |
| ACE054-01502 | 166.2(31.2)/235 | 20.7(0.98)/27 | |
| ACE054-02803 | 114.5(92.1)/270 | 22.4(2.9)/42 | |

The p35S-cry1Ab event gave absolute ECB control, both in the greenhouse and in field trials at different locations that correlated with the high-dose expression (as described above). Similarly, the pGos-gos-cry1Ab and PGos-cab-cry1Ab events displayed good ECB control at all locations tested. The pUbi-cry1Ab event did not show good ECB control.

No phytotoxicity was observed for any of the p35S-cry1Ab or pGos-cab-cry1Ab events. Selfed seed of pGos-gos-cry1Ab events showed segregation in the field of normal green plants and stunted, yellowish plants, from which the lower leaves are dying, suggestive of phytotoxicity.

Fourteen events obtained with the pTR2′-cry1Ab construct were evaluated for ECB efficacy in the greenhouse and in field trials (Table 5). Four of the five single-copy events (indicated with an asterisk) gave total ECB2 control.

TABLE 5
ECB Efficacy in the Greenhouse and in the Field
for pTR2′-cry1Ab events

| | ECB efficacy | ECB efficacy | ECB efficacy |
|---|---|---|---|
| | Greenhouse | Field | Field |
| | Average(sd)/max | average(sd)/max | Average(sd)/max |
| Event | tunnels/p1 | tunnels/p1 | tunnels/p1 |
| WI602-0402 | 0.1(0.31)/1 | 0.24(0.41)/2 | 0.21(0.01)/2 |
| W1604-1602 | 0.2(0.42)/1 | 0.05(0.071)/1 | |
| WI606-0406* | 51.3(73.5)/195 | 8.41(1.8)/32 | 7.45(0.07)/30 |
| WI606-0802 | 3.3(2.6)/8 | 0(0)/0 | 0.05(0.07)/1 |
| WI606-1206 | 0.38(0.74)/2 | 0.17(0.29)/3 | 0.1(0.14)/1 |
| WI600-0218 | 2.0(1.87)/6 | | |
| WI600-1402* | 29.4(45.8)/142 | 6.55(2.47)/28 | |
| WI602-0202* | 168.5(26.9)/200 | 12.6(3.8)/31 | |
| WI604-0604* | 102.5(83)/251 | 13.7(2.0)/25 | |
| WI602-0802 | 0(0)/0 | 0.33(0.15)/3 | |
| WI602-0204 | 66(68.2)/160 | | |
| WI602-1002 | 102.4(63.4)/215 | | |
| WI606-0602 | 45.5(52.8)/160 | | |
| WI600-0802 | 0.9(1.44)/4 | 0.06(0.11)/2 | 0(0)/0 |

No penalty on agronomic performance was observed for the plants after second selfing (ear to row) of the different TR2′ events in any of the locations tested.

e) Efficacy against Sesamia nonagroides

Five mid whorl corn plants obtained with the pTR2′-cry1Ab construct were each infested with 2 egg masses. Damage was scored after 14 days and number of larvae were counted. Damage rating were averaged over the five plants. Non-transformed B73 plants were similarly infested as controls.

| | Number of | Plant | |
|---|---|---|---|
| Event | larvae per plant | height in cm | Cm tunnels per plant |
| WI600-0802 | 0.6 (1.3) | 198 (8.4) | 0 |
| B73 Control | 197.2 (14.0) | 84 (5.5) | 70 (9.4) |

Example 3. Cry1Ab gene in cotton

I. Development of Cry1Ab events in cotton

A construct was made for the expression of the modified cry1Ab gene in cotton. The pTSVHO203 construct contains the modified cry1Ab coding sequence (encoding part of the Cry1Ab5 protein described by Höofte et al. 1986 having an insertion of an alanine codon (GCT) 3′ of the ATG start codon) under control of the constitutive promoters 35S (Odell et al. 1985) linked to the leader sequence of the tapetum El gene from Oryza sativa (W092/13956) with the 3′ ocs terminator (fragment containing polyadenylation signals from the 3′ untranslated region of the octopine synthase gene from the TL-DNA of pTiAch5, De Greve et al., 1982). The construct additionally comprises the bar coding sequence (the coding sequence of phosphinothricin acetyl transferase of Streptomyces hygroscopicus; Thompson et al., 1987) under control of the 35S promoter.

| | | |
|---|---|---|
| PTSVH0203 | P35S2-GE1-cry1Ab53-3′ocs<>p35S3- | p35S-cry1Ab-ocs |
| | bar-3′nos | |

This construct used for transformation of cotton. The obtained events were subjected to molecular analysis to confirm presence of the transgene.

II. Analysis of events

a) Expression of modified Cry1Ab

Expression of modified Cry1Ab protein was determined in leaf, terminal, square, flower and boll samples using ELISA (Table 7). Results represent percentage of Cry1Ab protein of total protein content as an average of samples taken from 9 plants. The time point at which samples were taken is represented as days after pollination (in brackets).

TABLE 7
Cry1Ab expression as measured by ELISA in different tissues

| | ELISA, Cry1Ab expression in %, 9 plants | | | | | | | | |
|---|---|---|---|---|---|---|---|---|---|
| | Leaf | Leaf | Leaf | Terminal leaf | Terminal leaf | Terminal leaf | Squares | Flower | Boils |
| Events | (60) | (65-80) | (115-120) | (60) | (65-80) | (115-120) | (60) | (65-80) | (115-120) |
| COCE040-04702A | 0.024 | 0.008 | 0.005 | 0.005 | 0.012 | 0.006 | 0.002 | 0.003 | 0 |
| COCE040-04702B | | 0.011 | | | | | | | |
| COCE040-3106-1143 | 0.017 | 0.022 | 0.012 | 0.015 | 0.011 | 0.007 | | 0.003 | |
| COCE040-3106-1144 | | 0.018 | | | | | | | |

b) laboratory toxicity assay

CBW larvae were fed on squares, leaf and terminals obtained from leaves in the field. Weight of surviving larvae was measured for the different events (Table 8). Samples from two non-transgenic plants and from one plant comprising a herbicide resistance gene were used as control.

TABLE 8
Toxicity of modified Cry1Ab expressed in cotton to CBW larvae

| | Weight in mg of surviving larvae | | |
|---|---|---|---|
| Event | Squares | Terminals | Leaf |
| 5 | 2.8 | 0 | 8.7 |
| 8 (−control LL25) | 22.3 | 1.7 | 15.5 |
| 11 | 0 | 0 | 18 |
| 13 | 4.3 | 1 | 20.6 |
| 16 | 6 | 0.3 | 23 |
| 21 (−control non transgenic) | 16.5 | 8.7 | 26.3 |
| 23 (−control non-transgenic) | 8.2 | 38.5 | 29 |

c) Efficacy of insect control in the field

In the field, plants were artificially infested with neonate CBW larvae. Damage to leaves, terminals, squares, white bloom and bolls was rated and counts were made each 8 days in August and September. A mean was made over the five observations dates. Statistical analysis indicated that significant differences were found in the mean damage severity of terminals, white blooms and bolls. In addition, the mean number of damaged squares and damaged bolls was higher for the controls than for the Cry1Ab events; The mean number of larvae in squares was also significantly reduced compared to controls.

Example 4. Cry1Ab gene in rice

A construct was made for the expression of the modified cry1Ab gene in rice. The construct contains the modified cry1Ab coding sequence (encoding part of the Cry1Ab5 protein described by Höfte et al.1986 having an insertion of an alanine codon (GCT) 3′ of the ATG start codon) under control of the promoter with the 5′ leader sequence of the GOS2 gene from rice, containing the second exon, the first intron and the first exon of the GOS transcript (de Pater et al., 1992). This construct used for transformation of rice. The obtained events were subjected to molecular analysis to confirm presence of the transgene.

Plants of 25 different events were tested for control of rice leaf folder, yellow stem borer, and pink stem borer by counting the number of surviving larvae. Damage to the plants was assessed as damage to the leaves (for leaf folder) or the number of deadhearts per number of tillers at the preflowering stage (yellow stem borer and pink stem borer) or the number of whiteheads per number of tillers at the flowering stage (yellow stem borer). For only 2 events plants were found on which some (5-7 out of 25) of the larvae survived. All plants of all other events showed 90-100% dead larvae. While the control plants were either completely dried up or showed many folded leaves when infested with rice leaf folder, none of the plants of the cry1Ab events showed any significant damage. Similarly, no deadhearts or whiteheads were detected for the plants of the cry1Ab events, infected with yellow stem borer or pink stem borer.

### References

Arvidson et al., 1989, Mol Microbiol 3:1533

Barton et al. 1987, Plant Physiol 85:1103-1109

Bogorad 2000, Trends Biotechn 18(6):257-263

Breitler et al. 2001, Mol Breeding 7: 259-274

Casas et al., 1995, Plant Breed Rev 13:235-264

Chen et al., 1994, Theor Appl Genet 88:187-192

Christensen et al. 1992, Plant Mol Biol 18(4):675-89

Christou, 1997, Plant Mol Biol 35(1-2):197-203

Cordero et al. 1994, Plant J 6(2):141-50

Cornelissen & Vandewiele, 1989, Nucleic Acids Research 17:19-23

Crickmore et al. 1996, ₂₈th Annual Meeting of the Society for Invertebrate Pathology, Society for Invertebrate Pathology, Bethesda Md., p 14

D' Halluin et al., 1992, Plant Cell 4: 1495-1505

Datta et al., 1999, Methods Mol Biol 111:335-347

De Block et al. 1989, Plant Physiol 914:694-701

De Greve et al., 1983, J Mol Applied Gen: 499-511

De maagd et al. 1999, Trends Plant Sci 4:9-13

de Pater et al., 1992, Plant J 2:837-844

Depicker et al., 1982, J Mol Appl Gen 1: 561-573

Duan et al., 1996, Nature Biotech 14:494-498

Dunwell, 1999, Methods Mol Biol 111: 375-82

Finer et al., 1999, Cur Top Micriobiol Inmunol 240:59-80

Franck et al. 1980, Cell 21: 285-294

Grochulski et al., 1995, J Mol Biol 254:447

Guevara-Garcia et al., 1998, Plant J 4(3):495-505

Guthrie, 1989, Plant Breed Rev 6:209-243

Harpster et al. 1988, Mol Gen Genetics 212:182

Hiei et al., 1997, Plant Mol Biol 35:201-218

Hofte and Whiteley, 1989, Microbiol Rev 53(2):242-55

Höfte et al., 1986, Eur J Biochem 161:273-280

Hood et al., 1986. J. Bacterial. 168: 1291

Jansens et al., 1997, Crop Science 37(5):1616-1624

Johnson et al., 1989, PNAS 86:9871

Komari et al. 1998, Curr Opin Plant Biol 1(2):161-165

Langridge et al. 1989, Proc Natl Acad Sci USA 86:3219-3223

Newell, 2000, Mol Biotechnol 16(1):53-65

Odell et al., 1985, Nature 313: 810-812

Potrykus 1990, Cyba Found Symp 154:198-208

Poulsen, 1996, Plant Breeding 115 :209-225

Sawahel et al. 1995, Biotechniques 19(1):106-110

Schnepf et al., 1998, Microbiol Mol Biol Rev 62:775-806

Thompson et al., 1987, EMBO J 6: 2519-2523

van der Salm et al., 1994, Plant Mol Biol 26:51

Vasil et al. 1999, Methods Mol Biol 111:349-358

Velten et al. 1984, EMBO J. 12:2733-2730

Wilbur and Lipmann, 1983, Proc Natl Acad Sci U.S.A. 80:726

Witowsky & Siegfried, 1997, PBI Bulletin 14-15

1. A DNA sequence encoding a modified Cry1Ab protein comprising the nucleotide sequence of SEQ ID NO: 2.

2. A chimeric gene comprising the DNA sequence of claim 1 under control of a plant-expressible promoter.

3. A recombinant vector comprising the chimeric gene of claim 2.

4. A transgenic plant cell comprising the chimeric gene of claim 2.

5. A transgenic plant comprising the plant cell of claim 4.

6. The transgenic plant of claim 5, which is selected from corn, cotton, or rice.

7. A seed of the plant of claim 6, which comprises the DNA sequence of SEQ ID NO: 2, encoding a modified Cry1Ab protein.

8. A method for protecting a plant from insects pests, comprising introducing into the plant the chimeric gene of claim 2.

9. The method of claim 8, wherein said insect pests are lepidopteran insect pests.

10. The method of claim 9, wherein said insect pests are selected from the group of European Corn Borer, Sesamia nonagroidesCotton bollworm, tobacco budworm, yellow stem borer, pink stem borer, and the rice leaf folder.


SEQUENCE LISTING (2 sequences)

SEQ ID NO: 1 - protein, 617 aa, Artificial sequence
  modified protein from Bacillus thuringiensis
MADNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYD

SEQ ID NO: 2 - DNA, 1854 bp, Artificial sequence
  sequence encoding modified protein from Bacillus thuringiensis
atggctgacaacaaccccaacatcaacgagtgcatcccctacaactgcctgagcaacccagaggtggaggtgctgggtggtgagaggatcgagaccggttacacccccatcgacatcagcctgagcctgacccagttcctgctgagcgagttcgtgcctggtgctggcttcgtgctgggactagtggacatcatctggggcatcttcggtcccagccagtgggatgccttcctggtgcagatcgaacagttaattaaccaaagaatagaagaattcgctaggaaccaagccatctctagactggagggcctgagcaacctgtaccagatctacgccgagagcttccgcgagtgggaggctgaccccaccaacccagccctgcgcgaggagatgcgcatccagttcaacgacatgaactctgccctgaccaccgccatcccactcttcgctgtccagaactaccaggtccctctcctgtctgtctatgtgcaagctgccaacctccatctcagcgtccttcgcgacgtgagcgtctttgggcagaggtgggggttcgacgctgccaccatcaacagccgctacaacgacctgacgcgtctgatcggcaactacaccgaccacgcagtgagatggtacaacactgggcttgagagggtctggggtcccgacagccgcgactggatcaggtacaaccagttcaggcgtgaactcactctcaccgtcttggatatcgtcagtctcttccccaactacgacagcaggacctaccctatccggactgtgagccagctgacccgcgagatctacaccaaccccgtgctggagaacttcgacggcagcttcaggggctctgcccagggcatcgagggcagcatccgcagcccccacctgatggacatcctgaacagcatcaccatctacactgacgcccacaggggtgagtactactggtctggccaccagatcatggcttctcccgtgggcttcagcggtcccgagttcaccttccccctgtacggcacaatgggcaacgctgccccacagcagaggatcgtggcccagctgggccagggcgtgtaccgcaccctgagcagcaccctgtacaggaggcccttcaacatcggcatcaacaaccagcagctgagcgtgctggatggcaccgagttcgcctacggcaccagcagcaacctgcccagcgccgtataccgcaagagcggcactgtggacagcctggacgagatcccaccccagaacaacaacgtgccccctaggcaggggttctctcatcgcctctcacacgtgagcatgttccgcagcggcttcagcaacagcagcgtgagcatcatcagggctcccatgttcagctggatccaccgcagcgctgagttcaacaacatcattccaagtagccagatcactcagatcccactcaccaagagcaccaacctgggctccgggactagcgttgtcaagggaccagggttcactggaggcgacatcctgaggaggaccagcccaggccagatcagcaccttaagggtgaacatcaccgctcccctcagccaacgctacagggtcaggatcaggtacgcttccaccaccaacctgcagttccacaccagcatcgacggcaggcccatcaaccagggcaacttcagcgccaccatgagcagcggcagcaacctgcagagcggaagcttccgcactgtgggcttcactaccccattcaacttctccaacggcagcagcgtgttcaccctgtctgcccacgtgttcaacagcggcaacgaggtgtacatcgacaggatcgagtttgtcccagctgaggtgaccttcgaagctgagtacgactga